(19) United States
(12) Patent Application Publication
(10) Pub. No.: US 2009/0235949 A1
(43) Pub. Date: Sep. 24, 2009

(54) TREATING LICE WITH GASEOUS COMPOUNDS IN AN AIRTIGHT SPACE

(76) Inventors: Menachem RITTERBAND, Ness Ziona (IL); Yinon Shlomi, Rehovot (IL)

Correspondence Address: Pearl Cohen Zedek Latzer, LLP, 1500 Broadway, 12th Floor, New York, NY, 10036, US

(21) Appl. No.: 12/473,058

(22) Filed: May 27, 2009

Related U.S. Application Data
(63) Continuation-in-part of application No. PCT/IL2008/000031, filed on Jan. 6, 2008.

(51) Int. Cl. A61K 8/18

(52) U.S. Cl. 132/202; 132/212; 132/200

(57) ABSTRACT

A system and a method for treating lice and nits on a head of a patient. The system includes a cap for defining an airtight space around the head and a container for storing a mixture of gases for treating lice, a gas release mechanism for delivering the mixture of gases into the airtight space as an aerosol, and a gas distribution device having plurality of gas delivery pins. The mixture of gases causes irreversible damage to essential mechanisms of the lice.

### CROSS REFERENCE TO RELATED APPLICATIONS

This application is a Continuation-in-Part of PCT/IL2008/000031 filed Jan. 6, 2008.

This application claims priority benefits from PCT/IL2008/000031 filed Jan. 6, 2008, which claims priority benefits from U.S. Provisional Application No. 60/878,351, filed on Jan. 4, 2007, herein incorporated by reference in its entirety.

### FIELD OF THE INVENTION

The present invention relates to the field of medical treatment systems, more particularly, to a system and method for treating an infestation of head lice.

### BACKGROUND OF THE INVENTION

Infestation of the human body by lice is an increasingly prevalent social and health problem in many countries, including the USA. Lice infest hundreds of millions of people each year. Lice are very small insects, about 2-3 mm in length. They deposit eggs either on a hair or fabric fiber and attach them firmly with a cement-like excretion.

The life cycle of lice comprises an egg stage, three nymphal stages, and an adult stage, and takes about one month. A female louse lays approximately 100 eggs, which are glued to hairs of the host. The eggs generally hatch in about six to ten days, depending on temperature. The nymphs, (the larval stage in insect development), and adults suck blood, causing disturbance (itching, also known as pruritus) and secondary infection. The empty shells remaining after the nymphs emerge from the eggs look like white grains of sand.

Lice, being insects, use tracheae for respiration. Tracheae are a system of internal tubes (invaginations of the cuticle) penetrating the insect's body, through which air diffuses or is being pumped directly to the body tissues. Within body cells, chemical respiration takes place in the mitochondria, where ATP is produced under oxygen consumption.

The common treatment methods against lice involve liquids or lotions. An apparatus and process for killing human and animal vermin, which discloses treatment of lice within a gas-tight garment by using a volatile liquid, specifically methyl-formate, that is spread on a pad inside the garment and evaporates inside the garment, is described in U.S. Pat. No. 2,033,357 of Riker et al. patented 1936.

A method and device for eradicating lice and nits form an infested area, particularly the scalp of a human being, comprises a plastic or rubber cap for covering the head of a human being, and means for filling the cap with an inner gas, such as carbon dioxide from a pressurized-gas cartridge, in order to suffocate the lice and the nits enclosed by the cap, is described in EP Patent Application No. 8663016.9, publication No. 022100a4 A2, of Scolnik et al. filed Oct. 30, 1986.

A formulation for controlling human lice, which discloses pediculicidal formulations comprising a spinosyn, or a physiologically acceptable derivative or salt thereof, and a physiologically acceptable carrier, and methods of controlling lice infestations in a human with these formulations are provided in International Application No. PCT/US1999/013925, Publication No. WO/2000/001347, of Snyder, filed Jun. 21, 1999.

A pediculicide composition, in particular, a composition comprising a sesquiterpene alcohol and a suitable carrier, is described in International Application No. PCT/AU2006/001720, Publication No. WO/2007/056813, of Found, filed Nov. 17, 2006.

A method and composition for controlling lice on an organism is described in International Application No. PCT/NL2000/000196, Publication No. WO/2000/057704, of Kussendrager et al. filed Mar. 23, 2000.

All of these applications are incorporated by reference for all purposes as if fully set forth herein.

Although the various breeds of human lice are related, each of them has specific characteristics with regard to habitat and feeding. For example, head lice are small hard-shelled ectoparasites which cling to hair shafts while feeding, mating and laying eggs. The louse must remain on the head or it will die within a short period of time. Head lice proliferate at an incredible rate. A louse is ready to mate and reproduce within 10 hours after hatching. Under ideal conditions, a female louse may produce up to 300 eggs in its lifetime. Ideal conditions include an adequate food supply, environmental temperatures from about 28° C. to about 32° C., and relative humidity of about 70% to about 90%.

The louse's hard chitinous exoskeleton serves as protection from external elements. Lice eggs (or ova) are similarly protected by a chitinous sheath surrounding the eggs and attached to the hair shaft. Although lice may be affected by the use of an insecticide, the eggs often remain resistant to attack. Thus, present art optimum treatment of a lice infestation includes both a pediculicide, which kills the adult lice, and an ovicide, which interrupts the development of the eggs.

Treatment for eliminating head lice traditionally included home remedies such as smearing mayonnaise, olive oil, hair pomade, or some other heavily viscous material about an infested scalp coupled with rigorous combing of the hair and meticulous removal of adult lice, nymphs, and nits. Though these home remedies do not kill head lice, the prevailing thought is that the viscosity of the material makes it hard for head lice to roam about the scalp, making for easy removal. Such home remedies are usually ineffective at controlling head lice due to the ability of the lice to revive rapidly once these materials are removed.

More effective treatments for eliminating head lice involve massaging the infested scalp with over-the-counter (OTC) topical creams containing active insecticides. Because of their potential toxicity to the human host, the use of these topical formulas is regulated by the FDA. Over-the-counter insecticides typically have pyrethrins or permethrin as active ingredients

Biologically active agents have been used for some time in attempts to control lice. For example, lindane (gammabenzene hexachloride), organophosphates (malathion), natural pyrethrins, and synthetic compounds known as pyrethroids (such as permethrin) have been used as pediculicides in lice treatment formulations. These agents however, have drawbacks. Lindane has a poor safety profile, and lice have developed resistance to it. Natural pyrethrin requires frequent follow-up treatments because it provides only short term residual action. Synthetic pyrethroids, although more effective against lice than natural pediculicides, are often more toxic to the subject being treated.

Strains of head lice have been identified worldwide which are resistant to all currently available topical treatments. Possible neural damage to the human host prevents raising the insecticide levels above the current threshold in an attempt to combat these newer treatment resistant head lice.

None of the prior art provides a sufficiently effective solution, and there is therefore a need for a system and a method for treating an infestation of head lice.

### SUMMARY OF THE INVENTION

The present invention teaches an effective solution for an infestation of head lice, including a system and method of use, which eliminates both the lice and their eggs by means of effective insertion of gas near the roots of the patient's hair, with the gas including toxic material or materials, and enabling a combined attack also including suffocating gases.

The system for treating lice and nits on a head of a patient includes a cap for defining an airtight space around the head and a container for storing a mixture of gases and compressed gases for treating lice. In addition, a gas release mechanism for delivering the mixture of gases into the airtight space is also provided. Specifically, the mixture of gases is selected so that it causes irreversible damage to at least one essential mechanism of said lice.

According to the present invention an effective concentrations of acidic vapors in general and Carboxylic acids in particular are applied. The invented treatment is based on the realization that live lice and their eggs are damaged by inhalation or absorption of reactive acidic gas or vapor. Hence the present invention includes compositions comprising of carboxylic acids and methods of their application to human subjects.

While prior art methods involve application of active ingredients in liquid or cream forms, the present invention specifies the application of carboxylic acids active ingredients in gas or vapor form. Moreover, for containment of said gas or vapor near the scalp, said gas or vapor application is introduced under a head cap covering the hair and relatively well sealed under the hair line by snug fit of an elastic perimeter of said head cap.

Folk art of applying various substances to the head for controlling head lice sometimes suggest applying a covering cap on the head after a shampoo, gel, or liquid is applied onto the scalp and hair. In contrast, the method, according to the present invention, demands prior covering of the head with a covering cap and then after introducing the gas or vapor composition comprising of carboxylic acid into the space under said cap. That introduction is preferably done via dedicated passages incorporated into said cap.

The process of evaporation in a closed container will proceed until there are as many molecules returning to the liquid as there are escaping. At this point the vapor is said to be saturated, and the pressure of that vapor (usually expressed in mmHg) is called the saturated vapor pressure. At room temperature, the saturated vapor pressure is the maximum partial pressure concentration at which the gas can be obtained in gaseous form.

The overall concentration of each of said carboxylic acids components in the applied gas or vapor compositions is selected to be on the one hand effective in reducing the survival of lice and/or eggs and on the other hand to be of tolerable irritability to the skin of human subjects. Effective concentrations satisfying the above constrains, in part from room temperature saturated vapor saturation pressure, can be selected for example from: between 1% to 5%, between 5% to 10%, between 10% to 20%, between 20% to 30%, between 30% to 40%, between 40% to 50%, between 50% to 60%, between 60% to 70%, between 70% to 80%, and between 80% to 90%.

In some embodiments, a mixture of more than one type of carboxylic acid is contained in the composition according to the present invention. An advantage of such a mixture is that, since each type of carboxylic acid is active in deactivating lice, and since the maximum gas concentration of each carboxylic acid is limited by its associated saturation partial pressure at room temperature, the use of a mixture of several carboxylic acids can produce an overall increase in the total vapor concentration of effective carboxylic acids active ingredients.

One type of gas or gas mixture may target the respiratory system by attacking the soft tissues or by elimination of oxygen from binding to the oxygen carrying protein of the lice.

One type of gas or gas mixture may target the respiratory system by attacking the soft tissues (for example vapors of acids such as acetic acid, hydrochloric acid and nitric acid), or by elimination of oxygen from binding to the oxygen carrying protein of the lice (for example carbon monoxide).

Other gasses targeting other targets such as the reproductive system (e.g. some of the organophospates), or nervous system (e.g. insecticides belonging to organochlorines, carbamates, and organophosphates) may be combined at the same time, before, or after treatment with the first gas mixture. The combination of gasses for targeting two or more different systems may improve the effectiveness of treatment.

In some embodiments treatment is done first with one type of gas and afterwards with another type of gas, for example, the lice are initially attacked with toxic gas which diminishes their resilience, and afterwards they are attacked with suffocating gas. This way, the use of toxic gas can be reduced.

According to the present invention, the systems of the lice that are attacked include: the respiratory system, by attacking mucous membrane tissue; the respiratory system, by attacking joints; the respiratory system, by disrupting the body's absorption of oxygen; the nervous system; and the reproductive system.

The dilution of the carboxylic acid active ingredient in the applied compositions may be in liquids such as water or in other gases such as air or water vapor, but these examples should not be taken as limiting.

It is a core principle of the present invention that, while the storage for of the compositions may be in liquid or compressed gas form, the release and application of said composition onto the proximity of the human head subject is in gas or vapor form.

Processes of creating a carboxylic acid gas or vapor from a storage compartment can be achieved in many forms, also including: reaction of high concentration acid with water, and compressed gas containers.

A particular benefit of the formulations is their effectiveness on both lice and eggs, and against louse species that have become resistant to currently used products.

Another benefit of the present invention is that there is no need for intensive washing of the hair after application of the treatment.

Carboxylic acids are known as a general family of chemicals representative members of which are selected from the group comprising of: formic acid (insect stings), acetic acid, propionic acid, butyric acid, valeric acid (valerian), caproic acid, enanthic acid, caprylic acid, pelargonic acid, capric acid, lauric acid, palmitic acid, and stearic acid.

Experiments have shown that the carboxylic acids most effective for use according to the present invention are formic acid and acetic acid.

Formic acid, HCOOH or CH₂O₂ is the simplest carboxylic acid.

Acetic acid, CH₃COOH, or C₂H₄O₂, also known as is an organic acid, is a simple carboxylic acid too, which is a water-soluble component of vinegar, and has been used, in liquid, as a folk remedy which dissolves the cement of lice eggs.

In some embodiments, a vapor mixture of carboxylic acid in air is first produced in a closed container of volume V_{con}. Said closed containers can be selected from the group of: bottles, bags, balloons, or other containers known in the art. According to the present invention, the gas from said container is to be delivered into the space between the head cap and the patient scalp. It is assumed that there is some desired predetermined vapor concentration Cᵥ of a selected carboxylic acid, in order to create an effective formulation according to the present invention.

The production for such vapors from solid or liquid forms of carboxylic acids can be done in the following production process: (a) A thin film or powder of a carboxylic acid is first enclosed between two strips or plates to create a “strip source”. The strip source is placed inside a container. (b) The quantity of carboxylic acid contained in the strip source is selected such that if it is evaporated within said selected container with volume V_{con} then it can create a vapor of said carboxylic acid with at least with the desired concentration Cᵥ. (c) The strip source is placed inside the selected container. (d) To create the carboxylic acid vapor at any prescribed or desired time, the two sides of the strip source are separated so as to reveal the included film or power of carboxylic acid to contact with the surrounding air within said container. (e) The container is then kept sealed at least for the amount of time that is required for sufficient evaporation of said carboxylic acid film or powder in the amount to create the desired concentration Cᵥ within said container.

According to an embodiment of the present invention there is provided a system for treating lice and nits on a head of a patient, the system including: a container for storing a predetermined volume mixture of gases for treating lice and nits; a mixture of gases for treating lice stored inside the container; an inlet tube having a first end and a second end, wherein the first end is operatively connected to the container; a cap operatively connected to the second end of the inlet tube; and a sealing means, for preventing gas flow from any space between the cap and the head, securely connected to the cap, wherein the mixture of gases comprises at least one gas that causes irreversible damage to at least one essential mechanism of the lice, wherein the amount of the mixture of gases stored in the container, when released outside the cap, is diluted by surrounding air such that the diluted mixture reaches the patient at non-toxic levels; and a gas distribution device operatively connected to the inlet tube, the gas distribution device including: a gas distribution device base, having an interior volume; and a plurality of gas delivery pins operatively connected to the gas distribution device base wherein each of the gas delivery pins has an outlet hole.

According to further features in the described embodiments the mixture of gases for treating lice and nits stored inside the container has an effective amount of an acidic active ingredient component.

According to still further features in the described embodiments the acidic active ingredient component is selected from the group consisting of: carboxylic acid, a derivative, and a salt thereof, associated with a physiologically acceptable carrier.

According to still further features in the described embodiments the acidic active ingredient component is selected from the group consisting of: hydrogen chloride, carboxylic acids, a product of a reaction of carboxylic acid with water, and a derivative or salt thereof, associated with a physiologically acceptable carrier.

According to still further features in the described embodiments the container is selected from the group consisting of: a container having a predetermined volume between 5 cc and 200 cc, a predetermined volume between 200 cc and 300 cc, a container having a predetermined volume between 300 cc and 500 cc, a container having a predetermined volume between 500 cc and 700 cc, a container having a predetermined volume between 700 cc and 1000 cc, a container having a predetermined volume between 1000 cc and 1500 cc, a container having a predetermined volume between 1500 cc and 2000 cc, and a container having a predetermined volume between 2000 cc and 3000 cc.

According to still further features in the described embodiments the carboxylic acid is selected from the group including of: formic acid, acetic acid, propionic acid, butyric acid, valeric acid, caproic acid, enanthic acid, caprylic acid, pelargonic acid, capric acid, lauric acid, palmitic acid, and stearic acid.

According to still further features in the described embodiments the mixture of gases comprises at least two different acidic active ingredient substances selected from the group consisting of: carboxylic acids, a product of a reaction of carboxylic acid with water, a derivative or salt thereof, associated with a physiologically acceptable carrier, and hydrogen chloride.

According to still further features in the described embodiments the two different acidic active ingredient substances are selected from the group consisting of: acetic acid, formic acid, a product of a reaction of acetic acid with water, and hydrogen chloride.

According to still further features in the described embodiments a concentration of one or more of the acidic active ingredient is a component of a compound, has partial pressure conditions, in terms of parts from saturated normal temperature and pressure, wherein the partial pressure has a minimum percentage value and maximum percentage value, and wherein the minimum percentage value and maximum percentage value are selected from the group consisting of: a minimum percentage value of 5% and a maximum percentage value of 10%, a minimum percentage value of 10% and a maximum percentage value of 20%, a minimum percentage value of 20% and a maximum percentage value of 30%, a minimum percentage value of 30% and a maximum percentage value of 40%, a minimum percentage value of 40% and a maximum percentage value of 50%, a minimum percentage value of 60% and a maximum percentage value of 70%, a minimum percentage value of 70% and a maximum percentage value of 80%, a minimum percentage value of 80% and a maximum percentage value of 90%, and a minimum percentage value of 90% and a maximum percentage value of 100%.

According to still further features in the described embodiments the system for treating lice and nits further including: a second container for storing a predetermined volume mixture of gases for treating lice and nits; a second mixture of gases for treating lice stored inside the second container; a second inlet tube having a first end and a second end, wherein the first end is operatively connected to the container, and wherein the second end is operatively connected to the cap.

According to still further features in the described embodiments the system for treating lice and nits further including: a unidirectional valve operatively connected to the cap.

According to another features in the described embodiments the container is a rigid container.

According to another features in the described embodiments the container is an elastic container.

According to an embodiment of the present invention there is provided a system for treating lice and nits on a head of a patient, the system including: a gas container for storing a predetermined volume gas, wherein the container is selected from a group consisting of a rigid container and an elastic container; a gas stored inside the gas container; an active material container having a first end and a second end, wherein the first end of the active material container is operatively connected to the gas container; an active material for treating lice and nits stored inside the active material container; an inlet tube having a first end and a second end, wherein the first end of the inlet tube is operatively connected to the second end of the active material container; a cap operatively connected to the second end of the inlet tube; and a sealing means, for preventing gas flow from any space between the cap and the head, securely connected to the cap, wherein the mixture of gases comprises at least one gas that causes irreversible damage to at least one essential mechanism of the lice, wherein the amount of the mixture of gases stored in the container, when released outside the cap, is diluted by surrounding air such that the diluted mixture reaches the patient at non-toxic levels; and a gas distribution device operatively connected to the inlet tube, the gas distribution device including: a gas distribution device base, having an interior volume; and a plurality of gas delivery pins operatively connected to the gas distribution device base wherein each of the gas delivery pins has an outlet hole.

According to an embodiment of the present invention there is provided a method for treating lice and nits on a head of a patient including: mounting on a head of the patient a gas distribution device, the gas distribution device includes a plurality of gas delivery pins; mounting a cap over the head of the patient; connecting an inlet tube part of the gas distribution device to an inlet part of a gas container: delivering, by means of the gas distribution device, a predetermined volume of a first gas mixture containing an effective amount of gas mixture for treatment of lice infestation; waiting at least a predetermined minimal duration time; and removing the cap from the head after the predetermined minimal duration time.

According to still further features in the described embodiments the method of further including: delivering, by means of the gas distribution device, a predetermined volume of second gas mixture containing effective amounts of gas mixture for treatment of lice infestation.

According to still further features in the described embodiments the first gas mixture further comprises at least 50 percent atmospheric air.

According to still further features in the described embodiments the first gas mixture further comprises acetic acid as an active ingredient.

Additional objects and advantages of the invention will be set forth in part in the description which follows and, in part, will be obvious from the description, or may be learned by practice of the invention.

### BRIEF DESCRIPTION OF THE DRAWINGS

The invention is herein described, by way of example only, with reference to the accompanying drawings, wherein:

FIG. 1 illustrates a first embodiment of a system for treating lice and nits, according to the present invention.

FIG. 2 illustrates a second embodiment of a system for treating lice and nits, according to the present invention.

FIG. 3 illustrates the first embodiment of a system for treating lice and nits, on a head of a patient, having two sets of rigid containers inlet tubes and unidirectional valves, according to the present invention.

FIG. 4 illustrates the first embodiment of a system for treating lice and nits, on a head of a patient, having two sets of rigid containers, unidirectional valves, and inlet tubes, according to the present invention, with both inlet tubes joining into a single inlet tube.

FIG. 5 illustrates a third embodiment of a system, for treating lice and nits, on a head of a patient, having a gas distribution device, according to the present invention.

FIG. 6a illustrates a bottom view of a gas distribution device, according to the present invention, upon which the section plane a-a is marked.

FIG. 6b is a cross sectional view a-a schematic illustration of an exemplary, illustrative embodiment of a hollow gas distribution device, according to the present invention.

FIG. 7a illustrates the first embodiment of a system for treating lice and nits, on a head of a patient, according to the present invention, in which the active material is separately stored within an active material container.

FIG. 7b shows two active material containers, according to the present invention.

FIG. 8 is a block diagram schematically illustrates a method of treating lice and nits on a head of a patient, according to some embodiments of the invention.

### DETAILED DESCRIPTION OF EMBODIMENTS

The present invention is of a system and a method for treating lice and nits on a head of a patient.

The principles and operation of a system and a method for treating lice and nits on a head of a patient, according to the present invention may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings.

Unless otherwise defined, all technical and scientific terms used herein have the same meaning as commonly understood by one of ordinary skill in the art to which this invention belongs. The materials, dimensions, methods, and examples provided herein are illustrative only and are not intended to be limiting.

The following list is a legend of the numbering of the application illustrations:
100a first embodiment of a system (for treating lice and nits)
100b second embodiment of a system (for treating lice and nits)
100c third embodiment of a system (for treating lice and nits)
11a rigid container
11b elastic container
12a inlet tube
12b gas release mechanism
13 cap (airtight)
14 hair
15a mixture of gases (for treating lice)
15b gas
15c active material
16 space (airtight)
17 sealing means
18 unidirectional valve
20 gas distribution device
21 gas distribution device base
22 gas delivery pin
23 outlet hole
24 interior volume (of the gas distribution device base)
30 active material container
31 cork
32 coil
33 sponge
300 head

As used herein the specification and in the claims section that follows, the term “effective amount of an acidic active ingredient component” and the like refer to quantity and qualities of an active ingredient component sufficient to exterminate lice and nits on a patient's head. These can be determined according to experimental results.

The disclosed invention describes a system and method for treating lice on a patient's head. The invention utilizes a cap for defining the space around the treated hair, and an airtight sealing of the space around the treated hair, to apply a gas for treating lice. Reducing the volume that is treated allows using a small amount of gas, such that in case of releasing the gas outside the cap, the patient faces no danger due to the strong dilution of the gas. Keeping the gas compressed in a container enables a rapid delivery of the gas into the airtight space.

Additional options for delivery of the gas also include use of a flexible gas container which is pressed manually, a gas container built as a cylinder and piston which is pressed manually, or a container including a pump which can be activated manually or by means of a small engine, which could be electric for example.

This specification is not intended in any way to limit the present invention to any specific method of applying pressure to the material in order to deliver it in gas form.

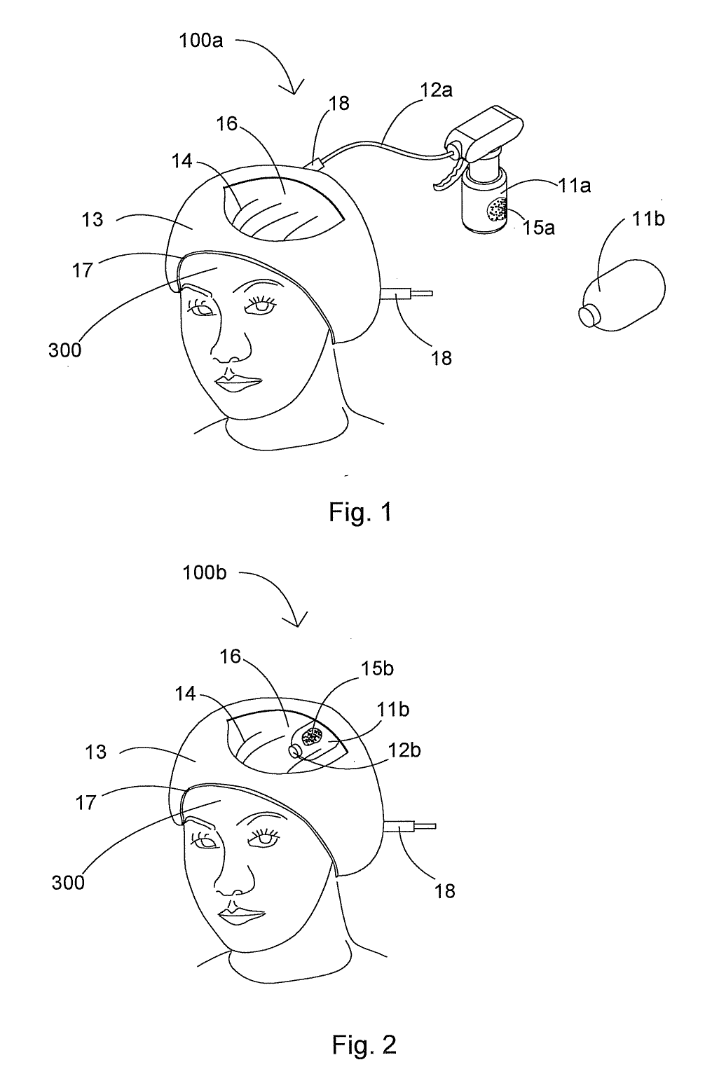

Referring now to the drawings, FIG. 1 illustrates a first embodiment of a system 100a, for treating lice and nits, on a head 300 of a patient, according to the present invention.

The first embodiment of a system 100a comprises an airtight cap 13 defining an airtight space 16 around head 300, a rigid container 11a for storing a mixture of gases 15a for treating lice and an inlet tube 12a for delivering mixture of gases 15a into airtight space 16 as an aerosol. Cap 13 is kept airtight by a sealing means 17 at the rim of cap 13. Mixture of gases 15a comprises at least one gas that causes irreversible damage to at least one essential mechanism of the lice. Rigid container 11a is located outside cap 13, and is coupled to cap 13 by inlet tube 12a, that comprises a tube.

If a segment of cap 13 is removed, and also if cap 13 is made of translucent material, the patient's hair 14 can be seen, as well as at least a part of airtight space 16, which is the volume between head 300 and hair 14, and the internal side of cap 13.

Airtight space 16 is not fixed, seeing as cap 13 can be made of material which is not rigid and therefore can change shape according to the regimen of pressures and forces applied to it.

Rigid container 11a contains a mixture of gases 15a which have a higher pressure than that of the environment, and thanks to the pressure difference, are delivered to the airtight space 16 in free flow, such as flow of fluid through an open tap. An additional option is storage of the mixture of gases 15a at a pressure of one atmosphere and delivering it when desired by means of activation of a pump or any other method which causes it to flow.

In some embodiments, rather than a rigid container 11a, there is an elastic container 11b, which contains the mixture of gases 15a. In this case, the delivery of gas is done by pressure applied to compress the elastic container 11b.

The first embodiment of a system 100a can also include a unidirectional valve 18 which enables flow of air and mixture of gases 15a out of the airtight space 16 when the pressure rises above a predefined value, as well as a unidirectional valve 18 assembled to the inlet tube 12a, preventing the return of the mixture of gases 15a from the airtight space 16, to the rigid container 11b or to the elastic container 11b, according to the type of container used.

The mixture of gases 15a can include various concentrations of toxic gases, suffocating gases, or combinations of toxic and suffocating gases.

Examples of possible types of gasses are: vapors of acids such as acetic acid, hydrochloric acid and nitric acid, for attacking the respiratory system by attacking the soft tissues; carbon monoxide, for attacking the respiratory system by preventing oxygen from binding to the oxygen carrying protein of the lice; organophospates, for attacking the reproductive system; and insecticides belonging to organochlorines, carbamates, and organophosphates for attacking the nervous system.

FIG. 2 illustrates a second embodiment of a system 100b, for treating lice and nits, on a head 300 of a patient, according to the present invention.

The second embodiment of a system 100b comprises of an airtight cap 13 defining an airtight space 16 around head 300, a container which can be a rigid container la or an elastic container 11b, for storing a mixture of gases 15a for treating lice and a gas release mechanism 12b for delivering mixture of gases 15a into airtight space 16. Cap 13 is kept airtight by a sealing means 17 at the rim of cap 13. Mixture of gases 15a comprises at least one gas that causes irreversible damage to at least one essential mechanism of said lice. The gas release mechanism 12b is a release valve contained within the container. The container is attached to the inner side of cap 13.

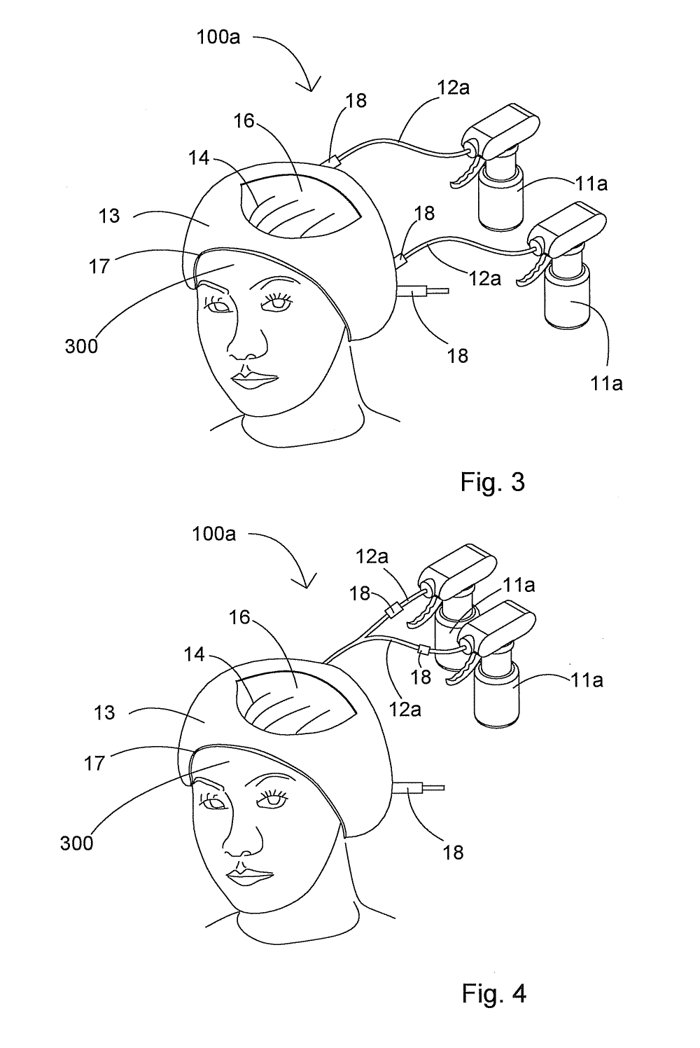

FIG. 3 illustrates the first embodiment of a system 100b, for treating lice and nits, on a head 300 of a patient, having two sets of rigid containers 11a, inlet tubes 12a, and unidirectional valves 18, according to the present invention. This configuration enables feeding two types of gasses or of mixtures of gasses into the airtight space 16 at the same time or at different times. When the feeding is done at different times, a suitable regimen of pressures can be used to press the first gas out of unidirectional valve 18 by means of the second gas. This valve is directed to enable only outward flow, while two additional unidirectional valves 18 are directed to prevent flow back into each one of both rigid containers 11a. Either of the rigid containers 11a can be replaced with an elastic container 11b.

FIG. 4 illustrates the first embodiment of a system 100b, for treating lice and nits, on a head 300 of a patient, having two sets of rigid containers 11a, unidirectional valves 18, and inlet tubes 12a, according to the present invention, while both of the inlet tubes 12a join into a single inlet tube, according to the present invention. This configuration also enables feeding two types of gasses or mixtures of gasses into the airtight space 16 at the same time or at different times. When the feeding is done at different times, a suitable regimen of pressures can be used to press the first gas out of unidirectional valve 18 by means of the second gas. This valve is directed to enable only outward flow, while two additional unidirectional valves 18 are directed to prevent flow back into each one of both rigid containers 11a. Either of the rigid containers 11a can be replaced with an elastic container 11b.

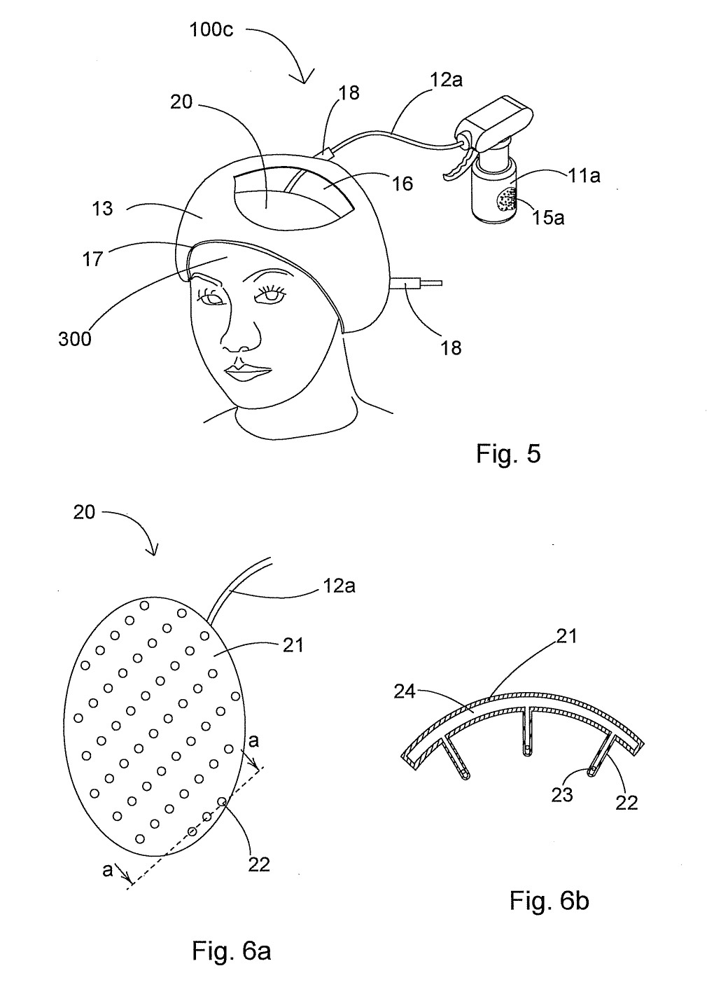

FIG. 5 illustrates the third embodiment of a system 100c, for treating lice and nits, on a head 300 of a patient, having a gas distribution device 20, according to the present invention.

The gas distribution device 20 serves for delivery and distribution under hair on a human scalp.

When gasses are intended to be used on human patient head as treatment of lice infestation, there are two functional problems:

(1) The gas needs to be prevented from escape away from around the head, and

(2) The gas needs to be preferentially applied in close proximity to the scalp where the lice mostly reside.

The solution to the first problem, as already outlined in the art, is to put a well-sealed cap over the head, and deliver the gas under the cap. Yet, as the gas is delivered from the top above the hair, only a minor fraction of it reaches under the hair in the proximity of the scalp skin (near the hair roots). Hence, such delivery of gas has a deficient effectiveness in treatment of lice infestation.

According to the present invention a gas distribution device 20 improve the effectiveness of gas treatments of lice infestations.

FIG. 6a illustrates a bottom view of a gas distribution device 20, according to the present invention, upon which the section plane a-a is marked. The present illustration shows a gas distribution device base 21, to the bottom of which are connected gas delivery pins 22.

FIG. 6b is a cross sectional view a-a schematic illustration of an exemplary, illustrative embodiment of a gas distribution device 20, according to the present invention.

The key element of the gas distribution device 20 is a series of gas delivery pins 22 with outlet holes 23 for gas or vapor delivery. Such gas delivery pins 22 may be short or elongated, stiff or flexible, with one or many outlet holes 23, etc.

Similar to the form of a brush, the gas delivery pins 22 are attached to and distributed over a common gas distribution device base 21. Here the gas distribution device base 21 has an interior volume 24. An inlet tube 12a is connected to the gas distribution device base 21. Hence, when vapor is pumped into the interior volume 24, through inlet tube 12a, it is then naturally fed into the connected gas delivery pins 22, and the gas is further on distributed out through the outlet holes 23. During use, this “brush” of pins is held in place pressed onto the scalp, such that the outlet holes 23 are under the hair in proximity to the scalp skin. Consequently, the gas is distributed as desired under the hair in proximity to the scalp skin.

In some embodiments, the end of said gas pins is rounded, and the outlet holes 23 are sideways from the shaft of the pins (as illustrated in the present figure).

The covering cap 13 is then put over the head and the gas distribution device 20, so as to prevent the delivered gas from escaping the proximity of the head. There are several options for engagement of a gas distribution device 20 in the third embodiment of a system 100c, for treating lice and nits. As was shown in FIG. 5, the inlet tube 12a, can go through the cap 13. In this case, the gas distribution device 20 and the cap 13 are a single unit and are placed together on the patient's head.

According to a second option, the gas distribution device 20 and the cap 13 are separated, the inlet tube 12a does not go through cap 13, and they are placed on the patient's head one after the other. In yet another option, the gas distribution device 20 comprises an integral part of the cap 13. Likewise, inlet tube 12a can be a continuous tube connecting the rigid container 11a, or the elastic container 11b, to the gas distribution device 20; or can be segmented, so that part of it is connected to the rigid container 11a, or the elastic container 11b, and its other part is connected to the gas distribution device 20, and only during application to the patient's head are they connected to each other to form a continuous inlet tube 12a.

According to some embodiments of the invention, the amount of mixture of gases 15a stored in the container, when released outside cap 13, is diluted by surrounding air such that the diluted mixture reaches non-toxic levels to the patient. The amount of mixture of gases 15a stored in the container is such that it would be highly toxic to the lice in a high concentration, and non-toxic to the patient if accidentally released into the surrounding air. The high concentration is achieved by defining and sealing the space 16 around the patient's head 300 and delivering mixture of gases 15a only into space 16.

According to some embodiments of the invention, the container holds mixture of gases 15a in a compressed state. Holding mixture of gases 15a in a compressed state allows a rapid release of mixture of gases 15a into airtight space 16.

According to some embodiments of the invention, cap 13 is a version of a swim cap, worn tightly on the patient's head. According to other embodiments of the invention, cap 15a is a version of a shower cap, worn loosely on the patient's head.

According to yet other embodiments of the invention, cap 13 is made in a version that is attractive in color and form to children. According to some embodiments of the invention, cap 13 is made of at least one of the following materials: silicone, rubber, polyethylene, latex, lycra, or cloth coated with a gas-tight layer.

According to some embodiments of the invention, exemplary composition of the gas mixture may include butane mixed with nitrogen, in a ratio ranging from 2:8 to 7:3 respectively. These gases are toxic.

According to some embodiments of the invention, the cap is configured to enable the mixture of gases to gradually flow outside of the cap to enhance safety.

According to some embodiments of the invention, exemplary duration of treatment ranges from 10 seconds to 30 minutes.

According to some embodiments of the invention, the cap 13 is configured to comprise small holes enabling a slow escape of the gas mixture, or alternatively the cap is made of a fabric made to allow slow gas escape of gas to the outside and air to the inside.

According to some embodiments of the invention, cap 13 is attached to the patient's head and sealed by sealing means 17 for preventing mixture of gases 13 from escaping out of cap 13. According to some embodiments of the invention, sealing means 17 comprises of a rubber band on the rim of cap 13. Sealing cap 13 provides a higher effectiveness of applied mixture of gases 15a as well as enhanced user safety.

According to some embodiments of the invention, mixture of gases 15a comprises a gas with low molecular weight and high diffusivity. These properties are needed to allow a quick penetration of the gas to airtight space 16, below hair 14 and into the tracheal system of the lice. According to some embodiments of the invention, mixture of gases 15a comprises a gas having a molecular weight below 100 Dalton, for example gaseous hydrocarbons, NH₃, SO₂, CO₂, CO.

According to some embodiments of the invention, mixture of gases 15a comprises a gas having a molecular weight below 1,000 Dalton, for example amino acids and peptides.

According to some embodiments of the invention, mixture of gases 15a depletes oxygen from airtight space 16 by physical or chemical means. Such means may be inert gases like N₂ that displace oxygen with airtight space 16 and within the tracheae of the lice. Alternatively the oxygen depletion may be chemically achieved by a reaction between at least one of the gases in the mixture and the oxygen in the airtight space.

According to some embodiments of the invention, a unidirectional valve 18 is attached to cap 13. The unidirectional valve is configured to allowing air out of cap 13 but preventing air from entering cap 13. The unidirectional valve 18 further reduces the airtight space 16 around the head of the patient, particularly depleting oxygen out of airtight space. Using the unidirectional valve also enhances the affectivity of mixture of gases 15a by concentrating the fixed amount of mixture of gases 15a in container 11 to a smaller space 16.

According to some embodiments of the invention, mixture of gases 15a causes irreversible damage to at least one essential mechanism of the lice, such as the mitochondria, nervous system, reproductive system, feeding system, etc. According to some embodiments of the invention, mixture of gases 15a comprises of specific inhibitors or destroyers of enzymes or reactions related to the proper functioning of these systems. According to other embodiments of the invention, mixture of gases 15a comprises of compounds preventing copulation or compounds that render nits sterile.

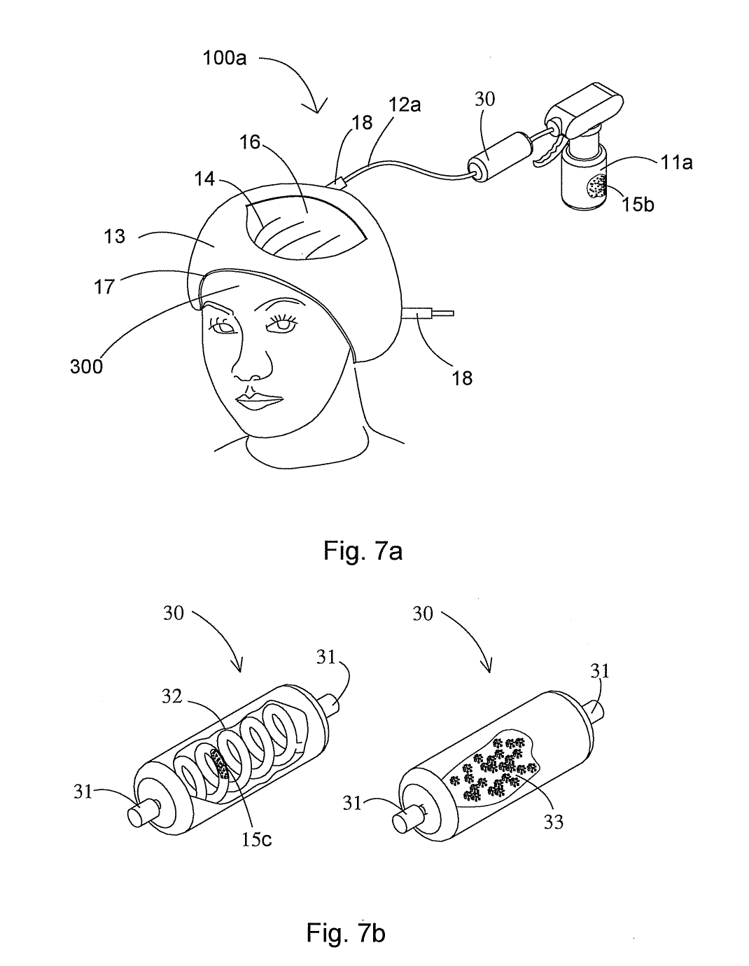

FIG. 7a illustrates the first embodiment of a system 100a, for treating lice and nits, on a head 300 of a patient, according to the present invention, in which the active material is stored separately within an active material container 30.

The active material container 30 is connected on both ends to inlet tube 12a. In this configuration, the rigid container 11a or elastic container 11b contains gas 15b at a sufficient pressure to maintain flow through the active material container 30 during use.

FIG. 7b shows two active material containers 30, according to the present invention. The right one contains a sponge 33 immersed with active material and the left one contains a coil 32 which contains an active material 15c. At both ends of the active material containers 30 shown here, there are two corks 31. This enables separate storage of the active material containers 30 containing active material 15c, and connection to inlet tube 12a prior to treatment of lice and nits.

The above description is not intended in any way to limit the present invention to these specific methods of storage and dispersion. This method of storing active material 15c separately from the carrying gas can be applied in any and all embodiments of the present invention.

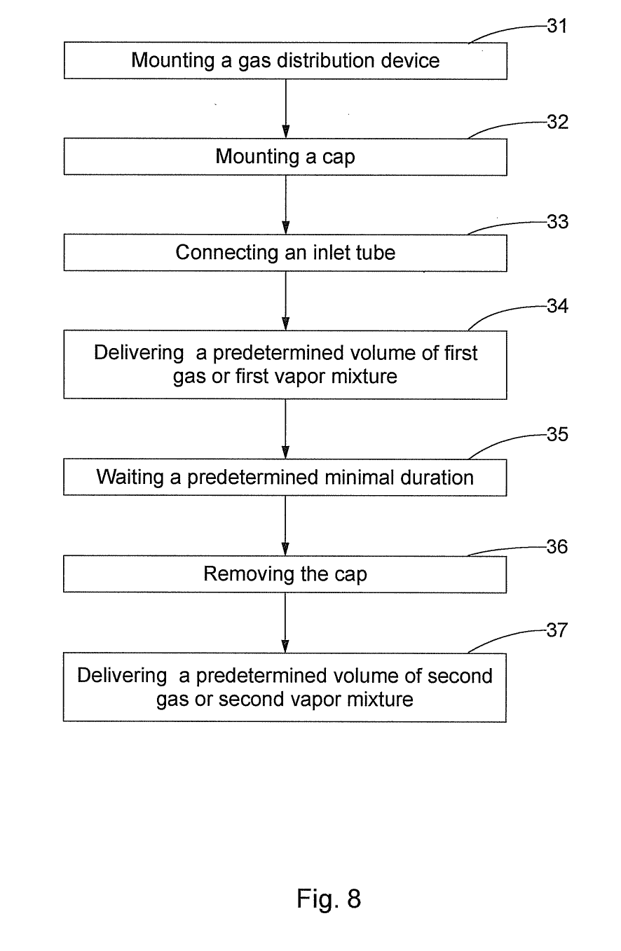

FIG. 8 is a block diagram schematically illustrating a method of treating lice and nits on a head of a patient, according to some embodiments of the invention.

The method includes:
(a) mounting on the head of the patient a gas distribution device, the gas distribution device including a plurality of gas delivery pins;
(b) mounting a cap over the head of the patient;
(c) connecting an inlet tube part of the gas distribution device to an inlet part of the gas container:
(d) delivering through the gas distribution device a predetermined volume of a first gas or first vapor mixture containing effective amounts of gas or vapor for treatment of lice infestation;
(e) waiting at least a predetermined minimal duration; and
(f) removing the cap from the head after the predetermined minimal duration.

The method can further comprise:
(g) delivering, through the gas distribution device, a predetermined volume of second gas or second vapor mixture containing effective amounts of gas or vapor for treatment of lice infestation.

In the above description, an embodiment is an example or implementation of the inventions. The various appearances of “one embodiment,” “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

It is understood that the phraseology and terminology employed herein is not to be construed as limiting and are for descriptive purpose only.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

It is to be understood that where the claims or specification refer to “a” or “an” element, such reference is not be construed that there is only one of that element.

It is to be understood that where the specification states that a component, feature, structure; or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described. Methods of the present invention may be implemented by performing or completing manually, automatically, or a combination thereof, selected steps or tasks. The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The descriptions, examples, methods and materials presented in the claims and the specification are not to be construed as limiting but rather as illustrative only.

Meanings of technical and scientific terms used herein are to be commonly understood as by one of ordinary skill in the art to which the invention belongs, unless otherwise defined.

The present invention can be implemented in the testing or practice with methods and materials equivalent or similar to those described herein.

Any publications, including patents, patent applications and articles, referenced or mentioned in this specification are herein incorporated in their entirety into the specification, to the same extent as if each individual publication was specifically and individually indicated to be incorporated herein. In addition, citation or identification of any reference in the description of some embodiments of the invention shall not be construed as an admission that such reference is available as prior art to the present invention.

While the invention has been described with respect to a limited number of embodiments, it will be appreciated that many variations, modifications and other applications of the invention may be made.

What is claimed is:

1. A system for treating lice and nits on a head of a patient, said system comprising:
(a) a container for storing a predetermined volume mixture of gases for treating lice and nits;
(b) a mixture of gases for treating lice stored inside said container;
(c) an inlet tube having a first end and a second end, wherein said first end is operatively connected to said container;
(d) a cap operatively connected to said second end of said inlet tube; and a sealing means, for preventing gas flow from any space between said cap and said head, securely connected to said cap, wherein said mixture of gases comprises at least one gas that causes irreversible damage to at least one essential mechanism of said lice, wherein the amount of said mixture of gases stored in said container, when released outside said cap, is diluted by surrounding air such that the diluted mixture reaches said patient at non-toxic levels; and
(e) a gas distribution device operatively connected to said inlet tube, said gas distribution device including:
(i) a gas distribution device base, having an interior volume; and
(ii) a plurality of gas delivery pins operatively connected to said gas distribution device base wherein each of said gas delivery pins has an outlet hole.

2. The system for treating lice and nits of claim 1, wherein said mixture of gases for treating lice and nits stored inside said container has an effective amount of an acidic active ingredient component.

3. The system for treating lice and nits of claim 2, wherein said acidic active ingredient component is selected from the group consisting of: carboxylic acid, a derivative, and a salt thereof, associated with a physiologically acceptable carrier.

4. The system for treating lice and nits of claim 2 wherein said acidic active ingredient component is selected from the group consisting of: hydrogen chloride, carboxylic acids, a product of a reaction of carboxylic acid with water, and a derivative or salt thereof, associated with a physiologically acceptable carrier.

5. The system for treating lice and nits of claim 2 wherein said container is selected from the group consisting of: a container having a predetermined volume between 5 cc and 200 c, a container having a predetermined volume between 200 cc and 300 cc, a container having a predetermined volume between 300 cc and 500 cc, a container having a predetermined volume between 500 cc and 700 cc, a container having a predetermined volume between 700 cc and 1000 cc, a container having a predetermined volume between 1000 cc and 1500 cc, a container having a predetermined volume between 1100 cc and 2100 cc, and a container having a predetermined volume between 2000 cc and 3000 cc.

6. The system for treating lice and nits of claim 3 wherein said carboxylic acid is selected from the group comprising of: formic acid, acetic acid, propionic acid, butyric acid, valeric acid, caproic acid, enanthic acid, caprylic acid, pelargonic acid, capric acid, lauric acid, palmitic acid, and stearic acid.

7. The system for treating lice and nits of claim 1, wherein said mixture of gases comprises of at least two different acidic active ingredient substances selected from the group consisting of: carboxylic acids, a product of a reaction of carboxylic acid with water, a derivative or salt thereof, associated with a physiologically acceptable carrier, and hydrogen chloride.

8. The system for treating lice and nits of claim 7, wherein said two different acidic active ingredient substances are selected from the group consisting of: acetic acid, formic acid, a product of a reaction of acetic acid with water, and an hydrogen chloride.

9. Any of the claims 2, 4, 7, or 8 wherein a concentration of one or more of said acidic active ingredient is a component of a compound, has partial pressure conditions, in terms of parts from saturated normal temperature and pressure, wherein said partial pressure has a minimum percentage value and maximum percentage value, and wherein the minimum percentage value and maximum percentage value are selected from the group consisting of: a minimum percentage value of 5% and a maximum percentage value of 10%, a minimum percentage value of 10% and a maximum percentage value of 20%, a minimum percentage value of 20% and a maximum percentage value of 30%, a minimum percentage value of 30% and a maximum percentage value of 40%, a minimum percentage value of 40% and a maximum percentage value of 50%, a minimum percentage value of 60% and a maximum percentage value of 70%, a minimum percentage value of 70% and a maximum percentage value of 80%, a minimum percentage value of 80% and a maximum percentage value of 90%, and a minimum percentage value of 90% and a maximum percentage value of 100%.

10. The system for treating lice and nits of claim 1, further comprising:
(f) a second container for storing a predetermined volume mixture of gases for treating lice and nits;
(g) a second mixture of gases for treating lice stored inside said second container;
(h) a second inlet tube having a first end and a second end, wherein said first end is operatively connected to said container, and wherein said second end is operatively connected to said cap.

11. The system for treating lice and nits of claim 1, further comprising:
(f) a unidirectional valve operatively connected to said cap.

12. The system for treating lice and nits of claim 1, wherein said container is a rigid container.

13. The system for treating lice and nits of claim 1, wherein said container is an elastic container.

14. A system for treating lice and nits on a head of a patient, said system comprising:
(a) a gas container for storing a predetermined volume gas, wherein said container is selected from a group consisting of a rigid container and an elastic container;
(b) gas stored inside said gas container;
(c) an active material container having a first end and a second end, wherein said first end of said active material container is operatively connected to said gas container;
(d) an active material for treating lice and nits stored inside said active material container;
(d) an inlet tube having a first end and a second end, wherein said first end of said inlet tube is operatively connected to said second end of said active material container;
(e) a cap operatively connected to said second end of said inlet tube; and a sealing means, for preventing gas flow from an space among said cap and said head, securely connected to said cap, wherein said mixture of gases comprises at least one gas that causes irreversible damage to at least one essential mechanism of said lice, wherein the amount of said mixture of gases stored in said container, when released outside said cap, is diluted by surrounding air such that the diluted mixture reaches said patient at non-toxic levels; and
(f) a gas distribution device operatively connected to said inlet tube, said gas distribution device including:
(i) a gas distribution device base, having an interior volume; and
(ii) a plurality of gas delivery pins operatively connected to said gas distribution device base wherein each of said gas delivery pins has an outlet hole.

15. A method for treating lice and nits on a head of a patient comprising:
(a) mounting on a head of the patient a gas distribution device, said gas distribution device includes a plurality of gas delivery pins;
(b) mounting a cap over the head of the patient;
(c) connecting an inlet tube part of said gas distribution device to an inlet part of a gas container:
(b) delivering, by means of said gas distribution device, a predetermined volume of a first gas mixture containing an effective amounts of gas mixture for treatment of lice infestation;
(e) waiting at least a predetermined minimal duration time; and
(f) removing said cap from the head after said predetermined minimal duration time.

16. The method of claim 15, further comprising:
(g) delivering, by means of said gas distribution device, a predetermined volume of second gas mixture containing effective amounts of gas mixture for treatment of lice infestation.

17. The method of claim 16, wherein said first gas mixture further comprises at least 50 percents atmospheric air.

18. The method of claim 16, wherein said first gas mixture further comprises acetic acid as an active ingredient.
